(19) United States
(12) Patent Application Publication
(10) Pub. No.: US 2022/0042986 A1
(43) Pub. Date: Feb. 10, 2022

(54) ELEVATED TEMPERATURE WASH FOR ELECTROPHORESIS

(71) Applicant: HELENA LABORATORIES CORPORATION, Beaumont, TX (US)

(72) Inventor: Tipton L. Golias, Beaumont, TX (US)

(73) Assignee: HELENA LABORATORIES CORPORATION, Beaumont, TX (US)

(21) Appl. No.: 17/303,509

(22) Filed: Jun. 1, 2021

Related U.S. Application Data
(60) Provisional application No. 63/063,446, filed on Aug. 10, 2020.

(51) Int. Cl. G01N 33/561

(52) U.S. Cl. CPC G01N 33/561

(57) ABSTRACT

An immunofixation electrophoresis method including separation of proteins (antigens) by electrophoresis, subjecting the separated proteins to various antisera to cause an antibody-antigen reaction and visualizing the results of the antibody-antigen reaction by staining, including a series of blotting and rehydrating steps with an elevated temperature rehydrating solution to remove unbound proteins (antigens), unreacted antisera, and excess stain.

### BACKGROUND OF THE INVENTION

The present invention relates to electrophoresis procedures in general and, in particular, to a method for enhancing the clarity of the results of an immunofixation electrophoresis procedure.

### Immunofixation Electrophoresis

Immunofixation electrophoresis, frequently referred to as IFE, is well-known as a two-stage or two-step procedure for detecting the presence of certain proteins in human serum, urine or cerebral spinal fluid. The procedure involves, as a first step, protein fraction resolution by electrophoresis. As a second step, the soluble antigen in the protein is allowed to react with an externally applied antibody (antiserum). The resultant antigen-antibody complexes will precipitate, at a rate dependent upon the proportion of the reactants, temperature, salt concentration and pH. The antigen-antibody complexes are then visualized by staining.

Typically, a specimen from a single patient is diluted and then placed in multiple sample or application areas, frequently referred to as zones or lanes, on a single electrophoretic gel plate. The gel plate may be an agarose gel, a polyacrylamide gel, or other suitable gel. The purpose of utilizing multiple sample areas for a single patient, is to enable detection, separately, of total serum protein, various proteins such as the immunoglobin heavy chains IgG, IgM, IgA and light chains Kappa and Lambda, or other proteins whose presence or absence may be of importance in medical diagnosis. As known in the prior art, various antisera (i.e., fluid containing the antibody) such as IgG, IgM, etc., are deposited on the appropriate lanes and permitted to react with the antigen in the sample.

The term “incubation” refers to the time interval during which the antisera and antibody are in contact such that a reaction may occur.

Improvements in the IFE procedure and equipment have progressed such that a single gel plate may accommodate not only multiple sample areas for a single patient, but also may accommodate multiple sample areas for multiple patients. Thus, if six zones or lanes are utilized for a single patient, and if a single gel plate accommodates nine patients, then there may be 54 lanes on the single gel plate.

After the electrophoretic separation step, the entire reaction zones or lanes must be covered with the appropriate antiserum since the antisera-antigen reaction or resolution, frequently referred to as the protein fraction resolution, may occur virtually at any position along the respective reaction zones. If the entire zone is not covered, depending on the location of the antigen in the patient sample, an antibody-antigen reaction may not occur. Therefore, covering the entire zone is important for qualitative purposes. Furthermore, there must be sufficient antiserum deposited such that all the antigen in the patient sample will react, otherwise the quantitative aspect of the test will be compromised. Therefore, it is conventional to apply excess amounts of antiserum.

After the incubation period, the relative percentage of the protein in each fraction or lane is obtained through the use of equipment such as a scanning densitometer. However, all the unreacted antisera and all unbound proteins should be removed prior to the qualitative and quantitative analysis otherwise, the scanning densitometer (or other equipment) may detect unreacted antisera and unbound proteins as noise, sometimes referred to as signal noise, leading to a lack of precision in the results.

To explain this further, consider, merely for illustrative purposes the IgG lane, i.e., the lane where the antisera for IgG is to be deposited. There must be excess antisera deposited to provide a reaction with all the IgG present in the sample. All the IgG in the patient sample should be bound to antisera for accurate results. After the incubation period, the excess antisera must be removed to eliminate noise. However, the patient sample included numerous proteins not just IgG and all the proteins are typically present in each lane. The proteins that did not bind to the antisera, referred to as the unbound proteins, must also be removed. In this example, all the unbound proteins in the IgG lane must be removed.

Historically, the unbound proteins and excess or unreacted antisera were removed by a multistep washing and heating procedure. As a non-limiting example, a typical procedure comprises a series of alternating blotting and rehydrating steps. The blotting steps remove excess antisera and unbound proteins, collectively referred to as fluid, from the gel plate and the blotting steps are carried out at elevated temperatures of approximately 50° C. (approximately 122° F.) and each blotting step had a duration of approximately five minutes. Between each blotting step, i.e., after each blotting step except the last blotting step, the unreacted proteins and excess antisera were rehydrated, such as with TBS, for approximately three minutes at room temperature of approximately 22° C. (72° F.). The blotting and rehydrating steps may be repeated three times (24 minutes) followed by a final blotting step (5 minutes) for a total of about 29 minutes.

After the removal of unbound proteins and unreacted antisera, a stain, such as but not limited to an acid violet stain is applied in each lane to visualize the result of the antibody-antigen reaction. The excess stain must then be removed following generally the same procedure of repetitive blotting at an elevated temperature and washing such as with TBS at room temperature for another 29 minutes.

Conventional wash (rehydration) at room temperature and drying (blotting) at elevated temperatures have been known for at least the last 75 years.

TBS refers to tris-buffered saline, a solution of approximately 40% Tris-HCl, 21% Tris Base, and 39% NaCl with a pH of 7.5. Tris is tris(hydroxymethyl)aminomethane.

### SUMMARY

The present invention relates to an improvement in the removal of unbound proteins, unreacted antisera, and excess, i.e., unbound stain by elevating the temperature of the rehydration solution contrary to conventional methods of removal.

### BRIEF DESCRIPTION OF THE DRAWINGS

In the drawings:

FIG. 1 is an illustration of a gel plate in which unbound proteins, unreacted antisera, and excess stain, have been removed according to conventional prior art practices; and

FIG. 2 is an illustration of a gel plate in which unbound proteins, unreacted antisera, and excess stain, have been removed according to the present invention.

### DETAILED DESCRIPTION

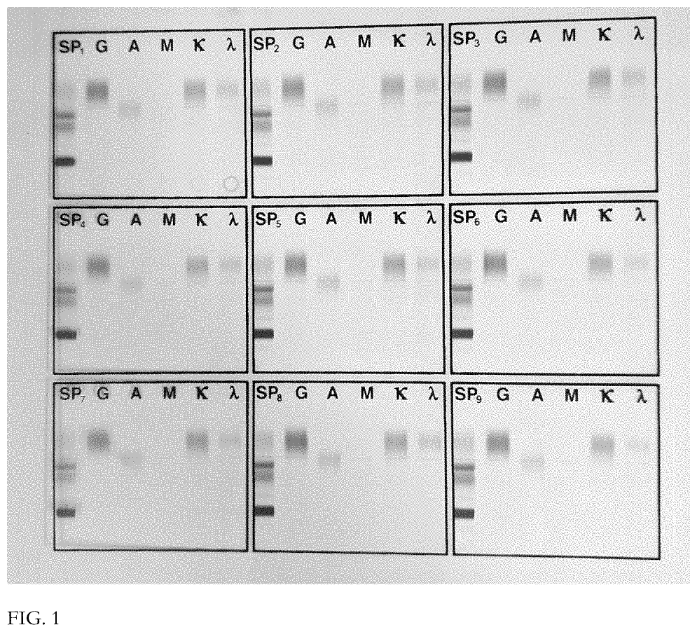

FIG. 1 illustrates a typical gel plate used in IFE in which unbound proteins, unreacted antisera, and excess stain, have been removed according to conventional prior art practices. The gel plate, which may be an agarose gel plate, may be used for evaluation of a single patient or for evaluation of multiple patients concurrently.

A typical gel plate may include areas for multiple patients such as in a 3×3 array such that nine patient samples may be electrophoresed concurrently. The areas for each patient may be generally square in shape and each patient area for an IFE procedure will include columns, referred to as lanes or zones, identified as SP (indicating total serum protein), G, A, M, κ (Kappa) and λ (Lambda).

Each patient area of the gel plate includes circular depressions or wells. Patient samples are placed in the wells and then subjected to electrophoresis. After electrophoresis, the antisera are applied to the zones for the immunofixation step. The immunofixation step is then followed by the washing and drying (rehydrating) as described above, to remove unbound proteins and unreacted antisera. A stain is applied for visualizing the results, and excess stain is then removed by washing and drying (rehydrating) as described above.

The nine patient samples on the IFE gel plate for FIG. 1 were processed using a conventional “wash” and “dry” procedure with TBS, that is, conventional blotting and rehydration times and temperatures as describe above with the rehydration occurring at room temperature, i.e., approximately 22° C. (72° F.).

In FIG. 1, the circular depressions or wells are visible at the bottom, and more particularly at the bottom right portion of the upper-left hand patient area. The reason is that within the depressions one or more of (a) unbound proteins, (b) unreacted antisera, and (c) excess stain are still present. FIG. 1 thus demonstrates the inconsistency among patient sample areas, i.e., some patient sample areas will not be sufficiently washed. Thus, a scanning densitometer (or other equipment) may detect unreacted antisera, unbound proteins and/or excess stain as noise, sometimes referred to as signal noise, leading to a lack of precision in the results.

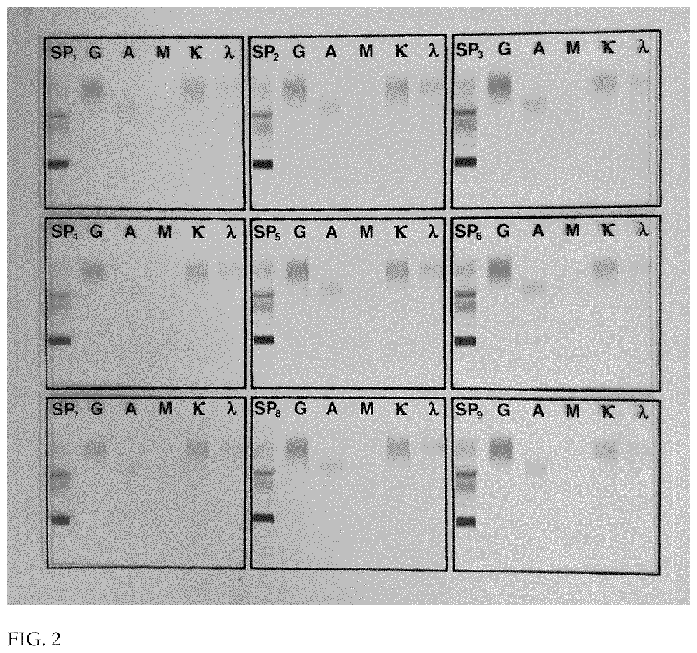

Referring next to FIG. 2, another electrophoresis plate is illustrated. The plate also has a 3×3 array of nine patient test areas, each within a generally square border, again with patient test area having zones or lanes SP (indicating total serum protein), G, A, M, κ (Kappa) and λ (Lambda). Each patient area has wells or depressions (not visible) for application of patient samples.

Using the same blotting and rehydration times and wash solution as described above for FIG. 1, but, contrary to conventional procedures, by elevating the wash or rehydration temperature to a range of about 30° C. to about 50° C. (about 86° F. to about 122° F.) there is a substantial decrease in the “background” in the lanes, i.e., a substantial decrease in unbound proteins, unreacted antisera and unbound stain.

Thus, the plate of FIG. 2, after the electrophoresis and immunofixation steps, was rehydrated at a temperature of 37° C. (98.6° F.). The various lanes or zones on the plate of FIG. 2 have a greater clarity in the region of the antibody-antigen reaction with more background or signal noise removed, when compared to the patient test areas on the plate of FIG. 1. This may be seen from a detailed lane-by-lane, patient-sample by patient-sample comparison since the patient samples and antisera and stain and the blotting and rehydration (washing) steps and times were identical as between the gel plate of FIG. 1 and the gel plate of FIG. 2 with the sole exception that the rehydration (wash) solution temperature was increased to 37 □C.

The difference as between the plate of FIG. 1 and the plate of FIG. 2 is more readily apparent when considering that the plate has wells or depressions that are not visible in the plate of FIG. 2. Thus, all the unbound proteins, unreacted antisera, and excess stain have been removed from the plate of FIG. 2 by the washing and elevated temperature rehydration steps. Thereafter, visualization of the results of the reaction will be more accurate and there will be substantially less signal noise.

The reason for the improved result of rehydration at an elevated temperature is not understood. Various hypotheses have been advanced. One hypothesis is that the elevated temperature rehydration denatures the non-reacted antisera, the non-reacted proteins (the antigen portion) in the patient sample, and the unbound stain, without damage to the bound antibody-antigen complex thus facilitating removal of these undesired components. A second hypothesis is that the elevated temperature rehydration causes thermal damage to the non-reacted antibodies, the non-reacted antigens, and the unbound stain, without damage to the bound antibody-antigen complex thus facilitating removal of these undesired components. A third hypothesis is that the elevated temperature rehydration expands the pores in the agarose gel upon which the IFE is performed to facilitate removal of the non-reacted antibodies, the non-reacted antigen, and the unbound stain, without damage to the bound antibody-antigen complex. Any two or all three hypotheses may be correct.

The foregoing is a complete description of the present invention for the improved rehydration of IFE gel plates.

What is claimed is:

1. In a method for removing unbound proteins and unreacted antisera from a gel plate having patient samples and antisera, the method including repetitive washing (rehydration) with a washing solution and blotting (drying) the gel plate, the improvement comprising facilitating removal of non-reacted antigens from the patient sample and non-reacted by rehydration with a washing solution having a temperature in the range of about 30° C. to about 50° C. (about 86° F. to about 122° F.).

2. The method of claim 1, wherein the washing solution has a temperature of about 37° C.

3. The method of claim 1, including one or more of:
a. denaturing any non-reacted antisera and/or any non-reacted antigen from the patient sample, without damage to the bound antibody-antigen complex formed by contact between the antibody and antigen;
b. thermally damaging any non-reacted antibodies and/or any non-reacted antigen from the patient sample, without damage to the bound antibody-antigen complex; and
c. expanding the pores in the gel upon which the IFE is performed to facilitate removal of any non-reacted antibodies and/or any non-reacted antigen, without damage to the bound antibody-antigen complex.

4. In a method for removing unbound proteins and unreacted antisera and unbound stain from a gel plate having zones for patient samples and antisera and stain in an IFE system, the method including electrophoresis of patient samples in the zones, contacting the electrophoresed patient samples in the zones with antisera causing reactions between the antisera and antigens in the zones, and application of a stain to the zones to bind to the antisera-antigen reaction, the method further including repetitive washing (rehydration) the gel plate with a washing solution and repetitive blotting (drying) the gel plate, the improvement comprising:
facilitating removal of non-reacted antigens from the patient samples on the gel plate and non-reacted antigens and unbound stain on the gel plate by rehydration with a washing solution having a temperature in the range of about 30° C. to about 50° C. (about 86° F. to about 122° F.).

5. The method according to claim 4, wherein the washing solution has a temperature of about 37° C.

6. The method of claim 4, including one or more of:
a. denaturing any non-reacted antisera and/or any non-reacted antigen from the patient sample, and/or any unbound stain, without damage to the bound antibody-antigen complex formed by contact between the antibody and antigen;
b. thermally damaging any non-reacted antibodies and/or any non-reacted antigen from the patient sample and/or any unbound stain, without damage to the bound antibody-antigen complex; and
c. expanding the pores in the gel upon which the IFE is performed to facilitate removal of any non-reacted antibodies and/or any non-reacted antigen and/or any unbound stain without damage to the bound antibody-antigen complex.
